(19) United States
(12) Patent Application Publication
(10) Pub. No.: US 2009/0069668 A1
(43) Pub. Date: Mar. 12, 2009

(54) METHOD AND MAGNETIC RESONANCE SYSTEM TO OPTIMIZE MR IMAGES

(76) Inventor: Alto Stemmer, Abenberg (DE)

Correspondence Address: SCHIFF HARDIN, LLP;PATENT DEPARTMENT, 6600 SEARS TOWER, CHICAGO, IL, 60606-6473, US

(21) Appl. No.: 12/203,283

(22) Filed: Sep. 3, 2008

(30) Foreign Application Priority Data
Sep. 3, 2007 (DE) 10 2007 041 826.6

(51) Int. Cl. A61B 5/055; G06K 9/00

(52) U.S. Cl. 600/413; 382/130

(57) ABSTRACT

In a method and magnetic resonance MR system for the optimization of angiographic MR images of an examination subject, in which arteries can be presented separately from veins in the angiographic magnetic resonance images, multiple MR overview images are acquired, with at least one imaging parameter being varied in the acquisitions of the MR overview images, at least one optimized imaging parameter is automatically calculated using a quality criterion, and the optimized imaging parameter is provided for the acquisition of the angiographic magnetic resonance images in which arteries can be shown separately from the veins.

### BACKGROUND OF THE INVENTION

1. Field of the Invention

The present invention concerns a method to optimize angiographic magnetic resonance (MR) images of an examination subject and a magnetic resonance system that implements such a method. The invention is particularly applicable in the generation of peripheral MR angiographs in which the angiographic images are generated without using a contrast agent.

2. Description of the Prior Art

One possibility to generate magnetic resonance angiographs without the use of contrast agents is the employment of fast spin echo imaging sequences, wherein a three-dimensional turbo spin echo imaging sequence is combined with a technique known as the half Fourier technique, for example. In the half Fourier technique, one half of Fourier space (domain) or k-space is not completely filled with measurement data, and the data that are not acquired are calculated by symmetry requirements of the data. Given suitable parameterization of the sequence, blood vessels are shown bright in such half Fourier turbo spin echo imaging sequences if the data acquisition ensues during a slow blood flow. By contrast, blood vessels appear dark if the blood flow was rapid during the signal acquisition.

It is important in magnetic resonance angiographies without usage of contrast agent to separate the arteries from the veins in the presentation of the blood vessels in the MR image. For this purpose, it is possible to synchronize the data acquisition with the cardiac cycle (and therefore the blood circulation), for example with the use of ECG triggering, and to acquire the MR data triggered by ECG. A first data set is thereby acquired during a heart phase in which the blood flow in the arteries and veins of the examination region is slow, which leads to both the arteries and the veins being shown bright in the image. When a second data set is to be acquired during a second phase of the cardiac cycle in which the blood flow is fast in the arteries of the examination region and is slow in the veins, the arteries appear dark in the associated angiography image and the veins are bright. In the following, the first phase in the blood circulation (in which the blood flow is slow in arteries and veins of the examination region) is called the diastolic phase (or diastole) and the second phase in the blood circulation (in which the blood flow is fast in the arteries of the examination region and slow in the veins) is called the systolic phase (or systole). Due to the time that the blood takes to flow from the heart into the examination region, the systole as defined herein generally occurs with a time delay relative to the contraction of the lower chamber of the heart muscle, which is commonly designated as cardiac systole. The same applies for diastole as defined herein. It is desirable to separate the artery information from the vein information. It should be possible to identify the veins from the angiographic images acquired during the systole. To identify the arteries it is necessary to subtract the MR data that were acquired during the diastole from the MR data that were acquired during the systole.

To suppress the signal portion of the surrounding tissue that is not flowing, it is possible to use a 180° pulse (inversion recovery pulse) before the actual image acquisition. For meaningful MR angiographic images and to separate the arteries from the veins, it is important to precisely find the diastole and the systole in the cardiac cycle and then to acquire MR angiographic images at these two points in time. Points of time are hereby discussed in connection with the present invention, wherein it is clear that neither the image acquisition nor the systoles and the diastoles are possible in an infinitesimally small time span. In U.S. Pat. No. 6,801,800 and Mitsue Miyazaki et al., “Non-Contrast-Enhanced MR Angiographic using 3D ECG-Synchronized Half-Fourier Fast Spin Echo”, Journal of Magnetic Resonance Imaging 12:776-783, 2000, it is described to acquire multiple ECG-triggered preparation acquisition images at different points in time of the cardiac cycle, wherein the different ECG-triggered images are displayed to the operator. The operator must now assess the images and find a first image in which the arteries and veins are presented as well as a second image in which the arteries are suppressed. The operator must then use the trigger delays that belong to the selected images in order to implement the MR angiographic. This procedure is very time-consuming and very error-prone. A specially trained and expert personnel is also necessary for the selection of the correct preparation images.

### SUMMARY OF THE INVENTION

An object of the present invention is to simplify non-contrast agent-enhanced MR angiography procedures insofar so that the correct imaging parameters can be determined in a simpler and quicker manner.

This object is achieved in accordance with the invention by a method for optimization of angiographic magnetic resonance images in which veins and arteries can be presented separately, wherein multiple MR overview images are acquired, and at least one imaging parameter is varied in the acquisitions of the MR overview images. At least one optimized imaging parameter is subsequently automatically calculated using a quality criterion, and the optimized imaging parameter(s) is/are provided for the acquisition of the angiographic magnetic resonance images in which arteries can be shown separately from the veins. In the method according to the present invention, the operator no longer needs to study MR overview images in order to determine the imaging parameter(s) with which arteries and veins can be separated. The operator is thus unburdened, the personnel do not need to be specially trained for this angiography method, and the residence time of an examination subject in the magnetic resonance system is shortened since an automatic determination of an optimized imaging parameter is distinctly faster and less error-prone than a manual determination by means of viewing multiple MR images.

According to a preferred embodiment, the imaging parameter is optimized to the extent that the angiographic magnetic resonance images are acquired during two different phases of the heart cycle to separate the arteries and veins. In this embodiment, “cardiac cycle” refers to the blood circulation since the blood flow speed is the decisive parameter. As mentioned above, it is advantageous to acquire MR angiography images during two points in time of the cardiac cycle since a signal difference between veins and arteries can be achieved given correct selection of the point in time. For this purpose, the MR overview images are advantageously acquired during various points in time of the cardiac cycle. Furthermore, the cardiac cycle is likewise advantageously monitored. One possibility for the optimized imaging parameter can be a trigger delay (TD). Naturally, the present invention is not limited to the optimization of a trigger delay. The present method can be used to optimize any other imaging parameters in such an angiography measurement. For example, it is also possible to optimize gradient circuits or, respectively, gradient amplitudes with the claimed method. It is likewise also possible to optimize more than one imaging parameter, wherein only one imaging parameter is optimized in a first step, for example, while the other imaging parameter to be optimized is kept constant during this first optimization. After the first imaging parameter has been optimized, in an additional step it can be sought to optimize the second imaging parameter, wherein it can be examined whether it is possible to further improve the quality criterion by optimization of the second imaging parameter. This optimization in two steps is generally quicker than the exhaustive search in a two-dimensional search region, but generally does not find the global optimum in the two-dimensional search region.

According to one embodiment, an optimized trigger delay TD_{Sys} for the acquisition of the angiographic MR image values is calculated during the systole and an optimized trigger delay TD_{Dias} for the acquisition of the angiographic MR images is calculated during the diastole. Through an optimized trigger delay, the imaging can be controlled such that the arteries and veins in the image are both shown bright once while another time only the veins are shown bright, such that images that essentially show only arteries are obtained via difference imaging.

In the variation of the imaging parameters in the acquisition of the various MR overview images, the trigger delay can be varied between a maximum value and a minimum value in order to generate the various MR overview images. The trigger delay is advantageously varied such that the entire cardiac cycle is covered with MR overview images.

As aforementioned, for non-contrast agent-enhanced MR angiography a fast, three-dimensional turbo spin echo sequence can be used in combination with the half Fourier technique. As used herein, “three-dimensional imaging sequence” does not refer to the successive excitation of multiple two-dimensional slices with a certain thickness, but rather means the excitation of the nuclear spins in a larger volume using a three-dimensional imaging sequence, with the resolution in the third dimension ensuing by an additional phase coding gradient as is typically the case in 3D acquisition techniques. In a fast half Fourier turbo spin echo sequence, all phase coding lines of a phase coding direction typically are measured along a single echo train while the moment of the phase coding gradients is constant for all echoes of this echo train in the other phase coding direction. The echo trains are then repeated for different moments of the other phase coding gradients.

It is desired to acquire the MR overview images with short acquisition time, and the sequence employed to acquire the MR overview images should optimally exhibit the same flow sensitivity as the sequence that is used to acquire the angiographic 3D MR data. According to the invention, one possibility to satisfy these requirements is to use an imaging sequence for generation of the MR overview images that essentially corresponds to the imaging sequence that is used for the angiographic 3D MR measurement, wherein, for the MR overview images, a phase coding gradient is deactivated in one of the two phase coding directions of the three-dimensional imaging sequence. Given use of a fast turbo spin echo sequence, for example, the echo train of the 3D sequence in which the phase coding gradient in the slice direction is zero is respectively switched to acquire an MR overview image. The imaging parameter to be optimized is varied between different MR overview images. The excited examination volume is projected onto a two-dimensional MR image via the use of a three-dimensional imaging sequence with deactivated phase coding in one direction.

The use of the three-dimensional excitation volume of which the angiography exposures should be acquired to generate a two-dimensional overview image is an important step for the continuing automation of the method since an extra positioning step to position the excitation volume for the overview images is omitted. For excitation of a thinner slice, as is typically the case in a two-dimensional measurement, it would first have to be ensured by the operator that the vessel to be presented is contained at all in the excited volume. The use of the three-dimensional imaging sequence with deactivated phase coding in one direction furthermore has the advantage that the same sequence scheme (and therefore the same flow sensitivity as for the subsequent actual angiography measurement) is used for the determination of the quality criterion. A 2D turbo spin echo sequence switches gradients (typically a few) that are necessary in order to suppress an unwanted signal from imperfect refocusing pulses, different than a 3D turbo spin echo sequence. It therefore also has a different flow sensitivity.

According to an embodiment of the invention, the multiple MR overview images can be subtracted in pairs from one another in order to generate difference images. These difference images can then be used as the basis for the calculation of the quality criterion. Using the difference images it can be detected whether the systolic cardiac phase and diastolic cardiac phase occurred in the overview images, since in this case only the arteries would have to be visible in the difference image since the veins have the same signal portion in both images while the signal portion of the arteries varies in the systolic phase and the diastolic phase, as was mentioned above.

According to a further embodiment of the invention, the MR overview images or the difference images can be masked or filtered. The goal of the masking or filtering is to avoid having to consider, or to consider to a lesser degree, pixels in overview images or difference images that are outside of a predetermined region. Dependent on the conoral orientation of the MR images, for example, the signal intensities at the upper and lower edges of the MR image in the direction of the body axis are typically subject to distortions. This is a consequence of the inhomogeneity of the B₀ field in this region. These distortions can lead to errors in the determination of the quality criterion. This is prevented by the masking or filtering of these regions.

The difference images are advantageously evaluated per pixel in the determination of the quality criterion, whereby each pixel can either be classified as “artery” or “background” or “undefined, for example. This is possible by the use of image segmentation algorithms and optionally with prior knowledge about the position and shape of the artery.

In the event that the calculation of the number of pixels that are classified as artery is greater than the number of the background pixels, these difference images are discarded, or the quality criterion is set to zero or to a lower value.

The quality criterion is a measure of how well the arteries in the difference images are able to be detected. One possibility to set the quality criterion is to determine an average signal difference between pixels that are classified as “artery” and pixels which are classified as “background”. If the average signal difference between “artery” and “background” is large, for example, it can be concluded that the difference image is of good quality, meaning that the artery is detected properly in the difference image. A value pair of the imaging parameters to be optimized is associated with each difference image via the MR overview images from which it was generated. As a result of the optimization, the value pair is now used that is associated with the difference image that maximizes the quality criterion. For example, if the trigger delay was varied as an imaging parameter in the acquisition of the MR overview images, two delay times are thus associated with each difference image. The difference image that maximizes the quality criterion now determines the two sought trigger delays TD_{Sys} and TD_{Dias}. TD_{Dias} is set equal to the trigger delay of its minuend and TD_{Sys} is set equal to the trigger delay of its subtrahend.

In an embodiment, it is possible to scan the cardiac cycle with a trigger delay change ΔTD in steps, such that the cardiac cycle is examined with different trigger delays that respectively differ by ΔTD within an R-spike (R-peak) interval. In another embodiment, a first optimization phase (run-through) is implemented in which the trigger delay TF is varied in larger steps, and from this first rough trigger delays TD_{Sys} and TD_{Dia} are calculated, while in a second optimization phase the trigger delays are varied in smaller steps and in a smaller search range in order to more precisely determine the trigger delays TD_{Sys} and TD_{Dia} determined in the first phase. Overall, the acquisition time to acquire the overview images can be shortened via the two-part optimization since overall fewer overview images must be acquired in comparison to the embodiment in which the cardiac cycle is examined in small trigger delay steps in one pass. In the prior art, a two-stage method would not lead to a reduction of the total examination duration, since the additional time that the operator requires to view the images after the first step and to determine the imaging parameters for the second step will generally be longer than the measurement time saved by the smaller total count of the overview images.

According to a further embodiment, a vessel enhancement filter is applied to the generated subtraction images in order to facilitate the image segmentation, for example. This vessel enhancement filter does not necessarily need to be applied. The arteries frequently can be sufficiently precisely identified even in the unfiltered difference images.

After the difference image or the two overview images that have led to an optimal contrast of the vessels have been identified with the use of the quality criterion, the calculated imaging parameters (in the present case the trigger delays TD_{Sys} and TD_{Dia}) can be displayed to the operator of the MR system. This operator can check the displayed values for plausibility and then use them in the subsequent three-dimensional MR angiography measurement. If the user interaction should be minimized further, it is possible to directly relay the calculated trigger delays directly to the image acquisition unit after the optimization. The image acquisition unit then automatically conducts the angiography measurements with the calculated trigger delays.

The invention furthermore concerns a magnetic resonance system for optimization of angiographic MR images of an examination subject, wherein arteries are presented separately from the veins in the MR images. The inventive MR system has an image acquisition unit to acquire multiple overview images, and an imaging parameter (such as the trigger delay, for example) is varied in the acquisition of the overview images. Furthermore, a calculation unit is provided that optimizes the imaging parameters using a quality criterion, and an output unit outputs the optimized imaging parameter. The optimized imaging parameter is either displayed on a display unit or directly passed to the image acquisition unit, which adopts the optimized imaging parameter and starts an angiographic MR measurement with this optimized value.

The invention furthermore concerns an electronically-readable data medium carrying control (programming) information that implements the method described above given use of the data medium in a computer system.

### BRIEF DESCRIPTION OF THE DRAWINGS

FIG. 1 schematically illustrates a magnetic resonance system for optimization of an angiographic measurement according to the invention.

FIG. 2 schematically shows a portion of the imaging sequence with simultaneous monitoring of the cardiac cycle.

FIG. 3 is a flow chart of an embodiment for parameter optimization in an MR angiographic measurement in accordance with the invention.

FIG. 4 is a flow chart with additional steps for parameter-optimized generation of MR angiographies in accordance with the invention.

### DESCRIPTION OF THE PREFERRED EMBODIMENTS

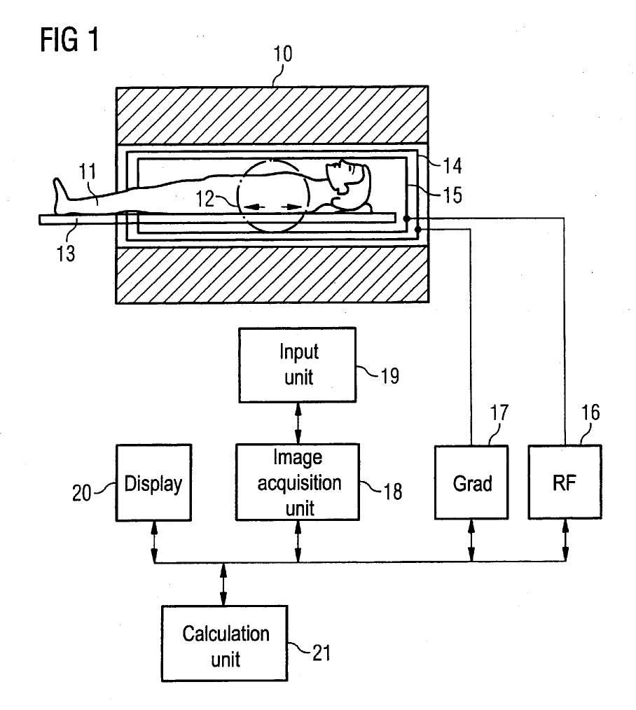

An MR system with which an imaging parameter can be optimized in a simple manner before conducting an angiographic measurement is schematically presented in FIG. 1. Such an MR system has a magnet 10 for generation of a polarization field B₀. An examination subject (here an examination subject 11) is moved on a bed 13 into the magnet 10, as is schematically depicted by the arrows 12. The MR system furthermore has a gradient system 14 for generation of magnetic field gradients that are used for the imaging and spatial coding. To excite spins that are polarized due to the basic magnetic field, a radio-frequency coil arrangement 15 is provided that radiates a radio-frequency field into the examination subject 11 in order to deflect the magnetization from the equilibrium (steady) state. A gradient unit 17 is provided to control the magnetic field gradients and a RF unit 16 is provided to control the radiated RF pulses. An image acquisition unit 18 centrally controls the magnetic resonance system; the selection of the imaging sequences likewise ensues in the image acquisition unit. The operator can select a sequence protocol via an input unit 19 and input and can modify imaging parameters that are displayed on a display 20.

The basic mode of operation of an MR system is known to those skilled in the art, so that a detailed description of the general components is not necessary. The MR system furthermore has a calculation unit 21 in which an imaging parameter can be automatically calculated and optimized in accordance with the invention.

The MR system shown in FIG. 1 can be used to generate angiography images by magnetic resonance. The present invention is concerned with non-contrast agent-enhanced angiography exposures. Such angiography exposures can be acquired with an imaging sequence, for example a half Fourier turbo spin echo sequence in which all phase coding lines in a phase encoding direction (for example k_{y}) are acquired during an echo train while in these three-dimensional imaging sequences the amplitude of the phase encoding gradients in the other phase coding direction (for example k_{z}) is the same for all echoes of this echo train. The echo trains are then repeated with the 90° excitation pulse and the refocusing pulses for various values of the phase coding gradients in the second phase coding direction (here k_{z}). In order to be able separate arteries from veins in non-contrast agent-enhanced MR angiography, according to one embodiment of the invention it is necessary to acquire the vessels during the systole and during the diastole of the cardiac cycle. In the diastole (the recovery phase of the heart), the blood speed in the arteries and veins is slow while the blood flow speed is fast in the arteries and slow in the veins during the systole (the contraction of the heart muscle). Such an imaging sequence typically can be implemented with monitoring of the cardiac activity with the use of an ECG (electrocardiogram). A 180° inversion pulse is typically used to suppress the background and the fat signal before switching the echo train, which 180° inversion pulse is temporally switched so that the background signals have an optimally small signal portion in the actual signal acquisition.

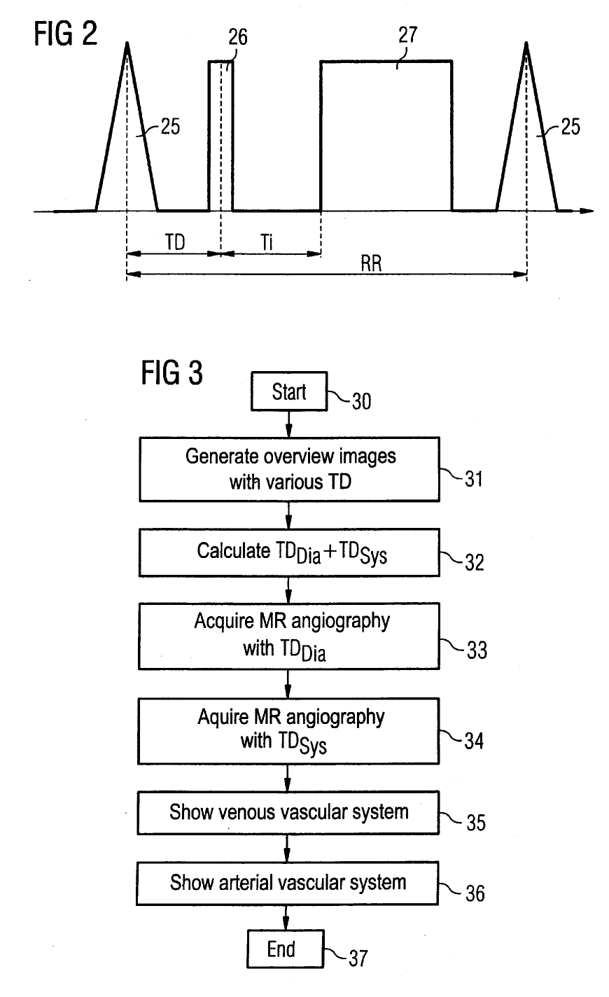

An excerpt from the imaging sequence is schematically presented in FIG. 2, wherein the cardiac activity is represented by the two R-spikes 25 of the ECG. After the detection of the R-spike in the ECG, the imaging sequence is triggered with a trigger delay TD. The start is an 180° inversion pulse 26, wherein the actual imaging sequence 27 ensues after the time span TI after this inversion pulse 26. This schematically represented imaging sequence 27 represents only a portion of the entire 3D imaging sequence, wherein only as many echo trains are read out as the heart rhythm allows before the remaining MR signals are acquired after a next R-spike. RR is the RR (spike) interval. It is also possible that only one echo train is read out in an RR interval, wherein it can be necessary to acquire image data only in every n-th (n=2, 3) RR interval in order to avoid a collapse of the signal.

For optimized MR angiographic images it is desirable to hit the point in time of the systole and the diastole in the RR interval in the signal acquisition. According to one embodiment of the invention, the delay TD is now varied in the acquisition of overview images in order to then be able to automatically calculate an optimized trigger delay TD_{Sys} for the systole and an optimized trigger delay TD_{Dia} for the diastole.

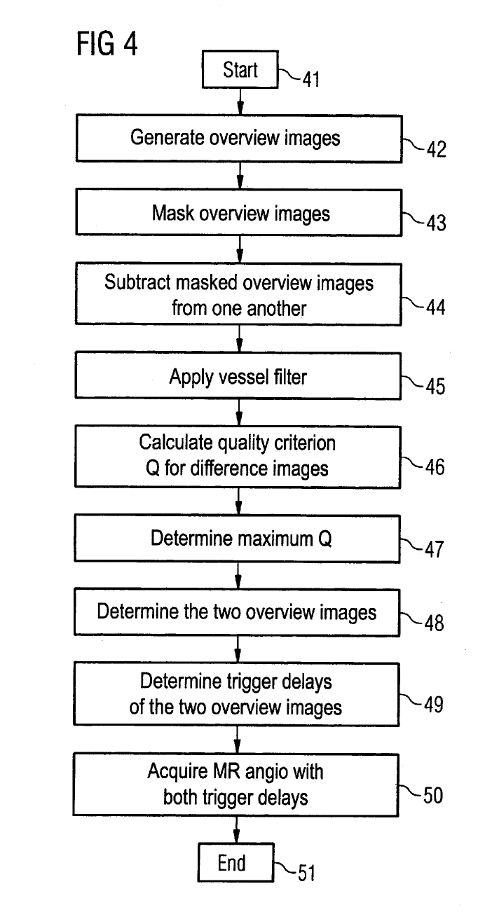

This optimization method is described in detail in connection with FIGS. 3 and 4.

After the start of the method in Step 30, in Step 31 various overview images are generated with various trigger delays TD. The number of overview images N is hereby adapted to the heart rate of the examined person, such that in total the entire cardiac cycle is covered. The different trigger delays TD hereby differ by ΔTD. As is easily recognized from FIG. 2, so many overview images must be generated that the following condition is satisfied:

N×ΔTD≧T_{RR} (1)

T_{RR} is the average time interval between two R-spikes. ΔTD can be selected between 50 and 100 ms, for example. The overview images are acquired with a three-dimensional half Fourier turbo spin echo imaging sequence, wherein the phase encoding gradient is set to zero in the second phase encoding direction. The entire excited volume is then projected onto a two-dimensional image with which, given correct positioning, it is ensured that the vessels to be presented are in each case contained in the overview images. Furthermore, a repositioning step is avoided for the acquisition of the overview images. As explained in detail in connection with FIG. 4, the generated overview images are evaluated using a quality criterion and the optimal trigger delays TD_{Dia} and TD_{Sys} are calculated (Step 32). These calculated trigger delays of the 3D volume can now be displayed to the operator on the display device 20 (for example TD_{Dia}=400 ms and TD_{Sys}=650 ms). The operator can then input these optimized imaging parameters into the imaging sequence via the input unit 19 so that the three-dimensional MR angiography images can subsequently be acquired with the optimized systolic and diastolic trigger delays (Step 33, Step 34). If an interaction with the operator is not desired or if the measurement workflow should be optimized further, it is also possible to pass the calculated, optimized trigger delays directly to the image acquisition unit 18, which then automatically conducts the three-dimensional MR angiography measurements. After the MR angiography measurements have been conducted, the venous vessels can be presented in one set of MR angiographic image images in a Step 35 and/or the arteries can be presented in Step 36. The method ends in Step 37. The method according to the invention has the advantage that an operator no longer needs to study the acquired overview images with the different trigger delays in order to obtain the optimized trigger delays.

The method according to the invention is shown again more precisely in FIG. 4. After a start in Step 41, overview images are generated, meaning a two-dimensional projection image of the acquired three-dimensional volume, wherein each overview image Iᵢ(x,y) signal possesses signals from resting tissue and from flowing tissue portions. The index i designates the number of overview images, wherein i runs from 1 to N. In these overview images the signal portion of the tissue that surrounds the vessels (what is known as the background signal) is high and the vessel is difficult to detect. The background signal is typically even relatively strong since a large amount of tissue contributes to the signal background with a slice thickness of multiple centimeters. The column index x with 1≦x≦Nₓ and the row index y designated with 1≦x≦N_{y} designate the spatial position of a pixel, wherein the x-axis runs along the readout direction and the y-axis runs along the first phase encoding direction. The trigger delay that is connected with each overview image Iᵢ(x,y) runs as follows:

TDᵢ=TD₁+(i−1)×ΔTD (2)

TD₁ is the trigger delay of the first overview image that can typically be set to zero. After all overview images have been generated in Step 42, the images can be masked in Step 43, which means that the values of pixel outside of a window are set to zero. In the event that x_{w}, y_{w} is the center of the window, wₓ is the length of the window in the column direction and w_{y} is the length of the window in the row direction, the pixel values after the masking are as follows:

$${I}_{i} ( x , y ) = \{ \begin{matrix}{I}_{i} ( x , y ) & {x}_{w} - \frac{{W}_{x}}{2} \leq x \leq {x}_{w} + \frac{{W}_{x}}{2} \\ 0 & otherwise\end{matrix} , \\ {y}_{w} - \frac{{W}_{y}}{2} \leq y \leq {y}_{w} + \frac{{W}_{y}}{2} \qquad ( 3 )$$

In a next Step 44, each masked overview image is then subtracted from every other overview image

S_{i,j}(x,y)=Iᵢ(x,y)−Iⱼ(x,y), i=1, . . . , N, j=1, . . . , N, i≠j (4)

This leads to N(N−1) new images in total, what are known as difference images or subtraction images. A vessel filter can optionally be applied to the generated difference images in Step 45; this vessel filter is not absolutely necessary. A quality criterion Q_{i,j} is calculated for each generated subtraction image in Step 46, wherein the quality criterion Q_{i,j} reflects the depiction of the arteries in the subtraction images S_{i,j}(x,y) (Step 46). The subtraction image that maximizes the quality criterion is now determined in Step 47. This means that the subtraction image with the highest quality criterion Q is selected. If the difference image with the best quality (i.e. with the best representation of the arteries according to the quality criterion) has now been determined, in Step 48 the overview images pair can be determined that has led to the difference image that had the best quality. With knowledge of the two overview images it is now possible to determine in Step 49 the associated trigger delays TD_{Sys} and TD_{Dia} that belong to the respective overview images. These optimized trigger delays can subsequently be used in Step 50 for the MR acquisition of the angiography before the method ends in Step 51.

Through the subtraction in Step 44, the background signal portions are reduced since the signal in unmoving tissue is typically the same for the different trigger delays. In this difference formation, every image is subtracted from every other image, which means that every image is a possible candidate for the optimal diastolic image and every image is a possible candidate for the optimal systolic image. After the subtraction step 44, there are generally three categories of subtraction images: The flow speed in the arteries is the same in both candidates, meaning the difference image typically contains essentially only noise. The flow speed can be significantly greater in the diastolic candidate than in the systolic candidate, so the arteries appear dark against the background. The flow speed is significantly greater in the systolic candidate than in the diastolic candidate image, so the arteries appear bright in comparison to the background and the veins appear dark since the vein speed does not change between systole and diastole. The last-mentioned category is the desired category.

For the calculation of the quality criterion, in one step it is established for each pixel of a difference image S_{i,j}(x,y) whether it is an artery pixel, a background pixel or an undefined pixel. The quality criterion of the difference image is then set equal to the difference between the average signal intensity of the artery pixels and the average intensity of the background pixels. In order to avoid an ambivalence in the order of the candidates, candidate pairs in which the number of artery pixels is greater than the number of background pixels are precluded. In the event that M_{i,j}(x,y) is a mask image that belongs to a difference image S_{i,j}(x,y), for the mask image the artery pixel N_{artery} can be set to 1, the background pixel N_{background} can be set to −1 and the undefined pixels can be set to 0. In this case, the quality criterion reads as follows

$$\begin{matrix}{Q}_{i , j} = \{ \begin{matrix}\frac{{\sum }_{y = 1}^{{N}_{y}} {\sum }_{x - 1}^{{N}_{x}} δ [ {M}_{i , j} ( x , y ) - 1 ] {S}_{i , j} ( x , y )}{{N}_{artery} ( i , j )} - \frac{{\sum }_{y = 1}^{{N}_{y}} {\sum }_{x - 1}^{{N}_{x}} δ [ {M}_{i , j} ( x , y ) + 1 ] {S}_{i , j} ( x , y )}{{N}_{background} ( i , j )} & {N}_{artery} ( i , j ) < {N}_{background} ( i , j ) \\ 0 & otherwise\end{matrix} & ( 5 ) \\ δ [ n ] = \{ \begin{matrix}1 & n = 0 \\ 0 & otherwise\end{matrix} & ( 6 )\end{matrix}$$

is hereby the Kronecker delta function.

$${N}_{artery} ( i , j ) = {\sum }_{y = 1}^{{N}_{y}} {\sum }_{x - 1}^{{N}_{x}} δ [ {M}_{i , j} ( x , y ) - 1 ] \qquad ( 7 )$$

is the number of pixels S_{i,j}(x,y) that were classified as artery and

$${N}_{background} ( i , j ) = {\sum }_{y = 1}^{{N}_{y}} {\sum }_{x - 1}^{{N}_{x}} δ [ {M}_{i , j} ( x , y ) + 1 ] \qquad ( 8 )$$

is the number of pixels S_{i,j}(x,y) that were classified as background.

A method for segmentation of the difference images is subsequently explained in detail. Segmentation thereby designates the classification of the pixels as an artery pixel, as a background pixel or as an undefined pixel. A technique known as the hysteresis threshold method can be used in the classification of the pixels of a difference image. This is a segmentation algorithm that is based on the fact that pixels that belong to an artery are connected with one another. The inputs for the segmentation algorithm are two thresholds Thresh_{low} and Thresh_{high}, with Thresh_{low}<Thresh_{high}. The algorithm surveys all pixels within the difference image. Each pixel with a signal intensity greater than or equal to Thresh_{high} that has not yet been classified is treated as a seed point (seed) for an artery. All seeds and all points with an intensity value greater than or equal to Thresh_{low} that are connected with the seed pixel directly or via other pixels with a value greater than or equal to Thresh_{low} are likewise classified as artery pixels. Furthermore, it is possible to implement a second pass of the segmentation algorithm in which all pixels that were not classified in the first pass, and that have a nominal interval smaller than a minimal interval DISTₘᵢₙ from a pixel that was classified as an artery pixel in the first pass, are classified as undefined. This second pass is implemented in order to avoid the dependency between the current values of the threshold parameters and the value of the quality criterion. Finally, all pixels that were classified neither as artery pixels nor as undefined pixels are classified as background pixels. Furthermore, the parameters Thresh_{low}, Thresh_{high} and DISTₘᵢₙ must be established. In general, hard experimental values cannot be used since the pixel values depend on the employed acquisition coils, the position of the coils on the patient and on many other factors. For example, the following prior knowledge about the arteries can be used in order to calculate the threshold parameters:
1. Whether the main artery direction runs along the x-direction or along the y-direction.
2. A rough estimation of the minimal thickness of a main artery can be established in units of pixels perpendicular to the main artery direction: TH_{artery}.

3. Furthermore, a prior knowledge about the number of main arteries in the image N_{arrtery} can be used.
4. The approximate length of the main artery in the image in units of the pixel size in the direction of the main artery L_{artery} can be established as prior knowledge.

In the event that the main artery direction lies along the y-axis, the following algorithm can used in order to calculate the threshold parameters.

Memory space is allocated for an array i_{artery} in that W_{y} integers can be stored and an integer variable Iₘₐₓ with the minimal integer value that can be presented by the computer is initialized.

For each row y of the image window it applies that the N_{artery}×TH_{artery} pixels of maximum intensity are to be found. The smallest of these values is used and stored in a position

y−(y_{w}−(W_{y}/2)ᵢₙₜ) (9)

of the array i_{artery}. The subscript int means that the value in parentheses is rounded down to the next whole number. The largest of these values is subsequently compared with Iₘₐₓ. If it is greater than Iₘₐₓ, the value of Iₘₐₓ is replaced by the largest value of the examined row.

After all rows of the image window have been processed, the values are sorted in ascending order in the array such that i_{artery}

i_{artey}[y₁]≦i_{artery}[y₂] (10)

Thresh_{low}=i_{artery}[W_{y}−L_{artery]}

Thresh_{high}=(Thresh_{low}+Iₘₐₓ)/2

DISTₘᵢₙ=TH_{artery} (11)

are subsequently ascertained.

If the main artery direction lies along the x-axis, a similar processing routine is used wherein the row index y is replaced by the column index x and the window size W_{y} is replaced by the window size Wₓ. Furthermore, the image window is processed column-by-column in the second step.

An image window must be defined for the masking of the overview images implemented in Step 43. This image window can be defined graphically by the operator during the slice positioning. The definition of the image window advantageously ensues automatically. Such angiography measurements in the extremities are typically implemented with a coronal alignment of the images and a large field of view. Greater magnetic field distortions typically occur at the edges of the image in the head-foot direction due to the B₀ field inhomogeneity in these regions. These regions can confuse the segmentation algorithm for classification of the pixels, such that these distorted pixels should lie outside of the image window. They following simple, automatic determination of the image window generally satisfies this requirement:

$${W}_{x} = \{ \begin{matrix}\frac{{N}_{x}}{2} & if the column direction points along the z \text{-} axis of the magnet \\ {N}_{x} & \end{matrix} \\ and \\ {W}_{y} = \{ \begin{matrix}\frac{{N}_{y}}{2} & if the row direction points along the z \text{-} axis of the magnet \\ {N}_{y} & \end{matrix} \qquad ( 12 )$$

A further possibility is the use of a vessel filter that enhances vessel-like structures of a specific direction and size in the image. Various such vessel filters are known in the prior art. These vessel filters can be used in order to improve the vessel segmentation.

One remaining aspect is the selection of the parameters main artery direction, TD_{artery}, N_{artery} and L_{artery}. It is possible to allow the operator select these parameters. According to another embodiment, however, these parameters are automatically selected, wherein the operator can naturally overwrite the selected parameters. In peripheral angiographies, the main artery direction most often runs in the foot-head direction of the examined person. If the readout gradient runs in the head-foot direction, the main artery direction runs in the column direction of the images; if the head-foot direction runs in the phase coding direction, the main artery direction runs in the row direction. The minimal artery thickness can be set to 5 mm, for example. The value TH_{artery} is then calculated in that 5 mm is divided by the pixel size in the direction perpendicular to the main artery direction. If both legs of the examined person are located in the field of view (as is typical), the number of main arteries N_{artery} can be set to 2, thus one for each leg. The length of an artery L_{artery} can be set equal to the unmasked window length along the primary direction of the artery. Naturally, a different selection of the parameters is possible. All of this information can improve the automatic determination of the arteries in the difference images.

In another embodiment of the invention it is furthermore possible to shorten the time for the acquisition of the overview images. The number of overview images that is acquired in order to cover one cardiac cycle is approximately N=T_{RR}/ΔTD. A typical RR interval has a length of T_{RR}=1000 ms if 60 heart beats per minute is the basis. A typical value for ΔTD is approximately 50 ms. In a turbo spin echo imaging, a measurement is possible only in every second or every third heart beat in order to acquire an acceptable signal. The total duration for the acquisition of the overview images is therefore T=N_{Trigger}×N×T_{RR}, wherein N_{Trigger} takes into account that image data can be acquired only every two (N_{Trigger}=2) or three (N_{Trigger}=3) heart beats. The acquisition duration at 60 heart beats per minute and measurement after every second heart beat is therefore: T=N_{Trigger}×N×T_{RR}=2×20×1000 ms=40 sec.

It is now possible to shorten this acquisition time in a multi-stage scanning method of the heart interval. In a first iteration, the distance ΔTD is increased so that only a rough sample of the RR interval ensues in a first iteration.

ΔTD^{rough}=2^{N}^{_{Iterations}}⁻¹ΔTD^{fine}

ΔTD^{fine} is the trigger delay change of the last iteration which determines the temporal resolution, and Nᵢₜₑᵣₐₜᵢₒₙₛ is the number of implemented iterations. The result of the first iteration is a first diastolic trigger delay TD_{Dia}⁽¹⁾ and a first systolic trigger delay TD_{Sys}⁽¹⁾. Delay ΔTD is halved relative to the preceding step in the second and every additional iteration. The previous, roughly determined delays can now be determined more precisely in the next step. The following delay times are executed for a more precise determination of the diastolic trigger delay

$${TD}_{1}^{( i )} = \{ \begin{matrix}{TD}_{Dia}^{( i - 1 )} - Δ T {D}^{( i )} & Δ T {D}^{( i )} \leq {TD}_{Dia}^{( i - 1 )} \\ {TD}_{Dia}^{( i - 1 )} + {T}_{RR} - Δ {TD}^{( i )} & Δ {TD}^{( i )} > {TD}_{Dia}^{( i - 1 )}\end{matrix} \qquad ( 13 )$$

TD₂⁽ⁱ⁾=TD_{Dia}⁽ⁱ⁻¹⁾+ΔTD (14)

The delay times are as follows for the systolic trigger delays

$$\begin{matrix}{TD}_{3}^{( i )} = \{ \begin{matrix}{TD}_{Sys}^{( i - 1 )} - Δ T {D}^{( i )} & Δ T {D}^{( i )} \leq {TD}_{Sys}^{( i - 1 )} \\ {TD}_{Sys}^{( i - 1 )} + {T}_{RR} - Δ {TD}^{( i )} & Δ {TD}^{( i )} > {TD}_{Sys}^{( i - 1 )}\end{matrix} & ( 15 ) \\ {TD}_{4}^{( i )} = {TD}_{Sys}^{( i - 1 )} + Δ {TD}^{( i )} & ( 16 )\end{matrix}$$

The overview images calculated using the four new trigger delays are masked, and eight new difference images are calculated. The quality criterion can subsequently be calculated for these eight additional difference images, wherein the calculated criteria can be compared with the result of the previous iteration. The maximum quality criterion is then selected as a result of the running iteration step. The last iteration step determines the total result. If, in such a two-stage method, the trigger delay is changed to ΔTD=100 ms in a first step and four additional measurements around the found trigger delays are implemented in a second step, the total acquisition time can be reduced to 28 seconds, for example, whereas it is approximately 40 s in a single-stage iteration with identical temporal resolution ΔTD=50 ms.

The invention has been described herein based on variation of the trigger delay in order to obtain an optimal trigger delay, but the present invention is not limited to the optimization of a trigger delay. With the method according to the invention it is also possible to automatically optimize other imaging parameters. For example, the flow sensitivity of the sequence can also be monitored via spoiler gradients of the turbo spin echo sequence, or additional gradients can be integrated into the sequence. The amplitude of such a gradient that leads to a best separation of arteries and veins can then be found automatically with the method according to the invention. The optimization of these additional parameters can ensue alone or together with the optimization of the trigger delay or in succession. In a successive optimization, in a first step one of the two parameters can be optimized while the other parameter is optimized in a second step.

The present invention enables the presentation of the veins separate from the arteries in a simple manner in non-contrast agent-enhanced angiography. The time-consuming and difficult selection of the overview images with the optimized imaging parameters of the arterial signal intensity given a variation of an imaging parameter that is known from the prior art can be foregone since the imaging parameters is automatically optimized. The measurement workflow is accelerated, such that the residence time of the examined person in the magnet can be shortened. Furthermore, specific training of the operator is not necessary.

Although further modifications and changes may be suggested by those skilled in the art, it is the intention of the inventors to embody within the patent warranted hereon all changes and modifications as reasonably and properly come within the scope of their contribution to the art.

1. A method for optimization of angiographic magnetic resonance images of a examination subject, comprising the steps of:
acquiring multiple magnetic resonance overview images of an examination subject wherein arteries and veins in the examination subject are represented and, during the acquisition of said multiple magnetic resonance overview images, varying at least one imaging parameter;
using said multiple magnetic resonance overview images, automatically calculating at least one optimized imaging parameter dependent on a quality criterion for representation of said veins and arteries; and
acquiring at least one angiographic magnetic resonance image from the subject using said optimized imaging parameter, in which said arteries are represented separately from said veins.

2. A method as claimed in claim 1 comprising optimizing said imaging parameter to acquire said angiographic magnetic resonance images, with said arteries and said veins shown separately, in two different phases of the heart cycle of the examination subject.

3. A method as claimed in claim 2 comprising acquiring said magnetic resonance overview images respectively at different points in time of the cardiac cycle.

4. A method as claimed in claim 1 comprising monitoring the cardiac cycle of the examination subject and optimizing a trigger delay as said optimized imaging parameter.

5. A method as claimed in claim 4 comprising varying said trigger delay between a maximum value and a minimum value during generation of said multiple magnetic resonance overview images.

6. A method as claimed in claim 4 comprising calculating a first optimized trigger delay for use in acquiring said angiographic magnetic resonance images during a first phase of the cardiac cycle, and calculating a second optimized trigger delay for acquisition of angiographic magnetic resonance images in a second phase of the cardiac cycle.

7. A method as claimed in claim 1 comprising employing an imaging sequence for acquisition of said multiple magnetic resonance overview images that corresponds to an imaging sequence employed for obtaining said angiographic magnetic resonance images, and activating a phase coding gradient in the acquisition of said magnetic resonance overview images in one of two phase coding directions of a three-dimensional imaging sequence.

8. A method as claimed in claim 1 comprising subtracting respective ones of said multiple magnetic resonance overview images from each other in pairs to generate a plurality of difference images, and calculating said quality criterion using said difference images.

9. A method as claimed in claim 8 comprising subjecting at least one of said overview images or said difference images to a signal processing procedure selected from the group consisting of masking pixels and filtering pixels, to cause pixels outside of a predetermined region to have a reduced contribution to calculation of said quality criterion.

10. A method as claimed in claim 8 comprising classifying pixels in said difference images respectively in categories selected from the group consisting of pixels representing arterial vessels, background pixels, and undefined pixels.

11. A method as claimed in claim 10 comprising calculating said quality criterion by calculating a difference between an average signal of pixels classified as representing an arterial vessel and an average signal of background pixels in said difference images.

12. A method as claimed in claim 11 comprising calculating a trigger delay as said optimized imaging parameter, and determining a first optimized trigger delay for acquiring said angiographic magnetic resonance images in a first phase of the cardiac cycle and determining a second optimized trigger delay for acquisition of the angiographic magnetic resonance images in a second phase of the cardiac cycle, and determining each of said first and second optimized trigger delays as the respective trigger delays associated with the two overview images whose difference image maximizes said difference between the average signal of arterial vessel pixels and the average signal of the background pixels.

13. A method as claimed in claim 1 comprising displaying said optimized imaging parameter to a user, and allowing the user to manually select imaging parameters for acquisition of said angiographic magnetic resonance images dependent on said optimized imaging parameters.

14. A method as claimed in claim 1 comprising automatically using said optimized imaging parameters to acquire said angiographic magnetic resonance images.

15. A method as claimed in claim 4 comprising varying said triggered delay in first steps in a first optimization phase and varying said trigger delay in second steps, smaller than said first steps, in a second optimization phase.

16. A method as claimed in claim 8 comprising applying a vessel enhancement filter to said difference images.

17. A method as claimed in claim 10 comprising rejecting difference images in which a number of pixels classified as representing arterial vessels is greater than a number of pixels that are classified as background, for use in calculation of said quality criterion.

18. A method as claimed in claim 10 comprising identifying said pixels representing arteries by post-processing of said overview images or said difference images.

19. A magnetic resonance system for optimization of angiographic magnetic resonance images of a examination subject, comprising:
an image acquisition unit that acquires multiple magnetic resonance overview images of an examination subject wherein arteries and veins in the examination subject are represented and, during the acquisition of said multiple magnetic resonance overview images, varies at least one imaging parameter;
a processor configured to use said multiple magnetic resonance overview images, to automatically calculate at least one optimized imaging parameter dependent on a quality criterion for representation of said veins and arteries; and
said processor making said optimized parameter available at an output thereof for use by said image acquisition unit to acquire at least one angiographic magnetic resonance image from the subject using said optimized imaging parameter, in which said arteries are represented separately from said veins.

20. A magnetic resonance system as claimed in claim 19 wherein said output unit is a display unit at which the optimized imaging parameters are visually presented.

21. A magnetic resonance system as claimed in claim 19 wherein said output unit transfers the optimized imaging parameter to said image acquisition unit, and wherein said image acquisition unit is configured to automatically acquire said angiographic magnetic resonance images using said optimized imaging parameter.

22. A computer-readable medium encoded with programming instructions for optimization of angiographic magnetic resonance images of an examination subject, said programming instructions causing a computerized control unit to operate a magnetic resonance imaging system to:
acquire multiple magnetic resonance overview images of an examination subject wherein arteries and veins in the examination subject are represented and, during the acquisition of said multiple magnetic resonance overview images, varying at least one imaging parameter;
use said multiple magnetic resonance overview images, automatically calculating at least one optimized imaging parameter dependent on a quality criterion for representation of said veins and arteries; and
acquire at least one angiographic magnetic resonance image from the subject using said optimized imaging parameter, in which said arteries are represented separately from said veins.
